(19) United States
(12) Patent Application Publication
(10) Pub. No.: US 2021/0346579 A1
(43) Pub. Date: Nov. 11, 2021

(54) HANDS-FREE MILK EXTRACTION AND COLLECTION DEVICE

(71) Applicant: AMANDA QUARLES, HOMESTEAD, FL (US)

(72) Inventor: AMANDA QUARLES, HOMESTEAD, FL (US)

(21) Appl. No.: 16/868,214

(22) Filed: May 6, 2020

(51) Int. Cl. A61M 1/06

(52) U.S. Cl. CPC A61M 1/064

(57) ABSTRACT

A hands-free milk extraction and collection device including a combined milk extraction and collection container and a housing unit. The container includes an internal cavity having an extraction portion for receiving a nipple region of a woman's breast and having a collection portion for collecting milk extracted from the woman's breast. The extraction portion is configured to latch onto the nipple region of the woman's breast to aid in stimulating milk flow from the breast by creating suction pressure at the nipple region. The housing unit includes a retention device for retaining the container in the housing unit, wherein the housing unit and the enclosed container are collectively configured to fit inside a woman's bra for hands-free usage.

### CROSS-REFERENCE TO RELATED APPLICATIONS

This application is a continuation-in-part of non-provisional utility patent application Ser. No. 16/268,496, filed Feb. 6, 2019, and entitled A HANDS-FREE MILK EXTRACTION AND COLLECTION DEVICE, the entire contents of which are hereby incorporated by reference.

### FIELD OF THE INVENTION

Embodiments described herein generally relate to a milk collection device, and more particularly to a hands-free milk extraction and collection device.

### BACKGROUND

Many women utilize a breast pump to induce milk lactation, which is stored and later bottle fed to their children. Although breast pumps allow mothers to conveniently prepare nursing bottles before feeding hours and/or serve to help mothers alleviate full breasts, most machines are hand-held and too large, making milk collection a time-consuming task that may also make some individuals feel uncomfortable while doing so while on-the-go. Hence, it is desirable to provide an improved milk collection device.

### SUMMARY

Exemplary embodiments disclosed herein describe a hands-free milk extraction and collection device including a milk collection container and a housing unit. The milk collection container includes an internal cavity having a first section for receiving a nipple region of a woman's breast and having a second section for collecting milk extracted from the woman's breast.

The housing unit is configured to latch onto the nipple region of the woman's breast to aid in stimulating milk flow from the breast by creating suction pressure at the nipple region. The housing unit having a shape contoured to a woman's breast and including a center opening for enclosing the milk collection container, wherein the housing unit and the enclosed milk collection container are collectively configured to fit inside a woman's bra for hands-free usage.

In some exemplary embodiments, the suction pressure at the nipple region is created without the use of an electric pump and without any manual pumping.

In some exemplary embodiments, the latching onto the nipple region of the woman's breast creates a seal around the nipple region.

In some exemplary embodiments, the housing unit latches onto the nipple region in a close-fitting manner.

In some exemplary embodiments, the suction pressure is created by the latching which effectuates squeezing of the nipple region of the breast, which causes extraction of milk from the breast.

In some exemplary embodiments, the device includes a plastic cap which is configured to fit into the woman's bra to keep the pressure from the bra from releasing the suction.

In some exemplary embodiments, the housing shell is a plastic shell.

In some exemplary embodiments, the milk collection container is plastic.

In another embodiment, the housing unit includes a retention device, e.g., a clip, to engage with a neck portion of a combined extraction and collection container in order to retain the container securely in the housing unit during operation.

### BRIEF DESCRIPTION OF THE DRAWINGS

The various advantages of the embodiments of the present disclosure will become apparent to one skilled in the art by reading the following specification and appended claims, and by referencing the following drawing(s), in which:

FIG. 1 shows an exemplary view of a hands-free milk extraction and collection device according to an embodiment of the present disclosure.

FIG. 2 shows another exemplary view of the hands-free milk extraction and collection device according to an embodiment of the present disclosure.

FIG. 3 is a perspective view of another embodiment of a hands-free milk extraction and collection device.

FIG. 4 is a rear perspective view of the embodiment shown in FIG. 3.

FIG. 5 shows the combined extraction and collection container separated from the housing unit.

FIG. 6 is a cross-sectional view of the device.

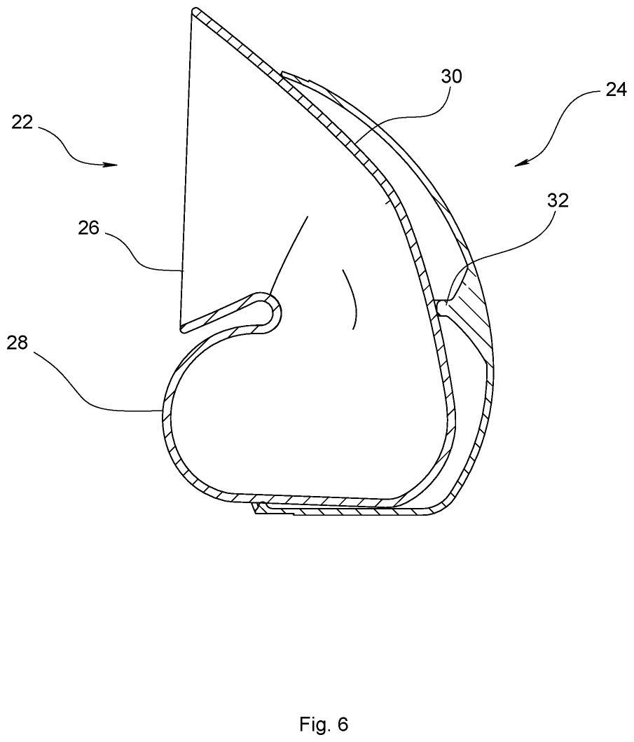

FIG. 7 shows an individual view of the combined extraction and collection container.

FIG. 8 shows an individual view of the housing unit.

FIG. 9 shows a user using the device.

### DETAILED DESCRIPTION

The present disclosure relates to a hands-free milk extraction and collection device. The device may fit into a nursing mother's bra for support. The device includes a suction mechanism that latches onto the nipple, which helps stimulate milk flow when milk let down occurs on the opposite breast (i.e., the non-nursing breast) while nursing. The milk will be collected in a milk collection container and can be poured for storage when the mother is done breastfeeding. The suction enables the device to not require electronic or manual pumping as the milk let down is a natural component of breastfeeding.

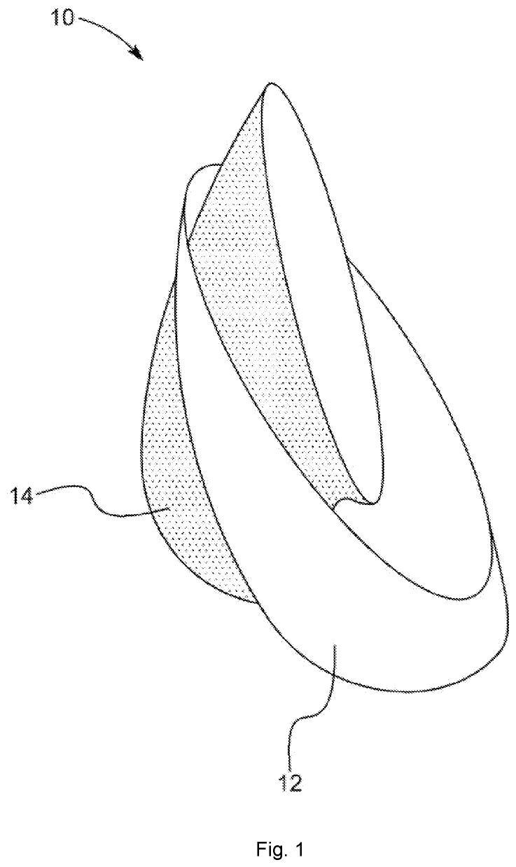

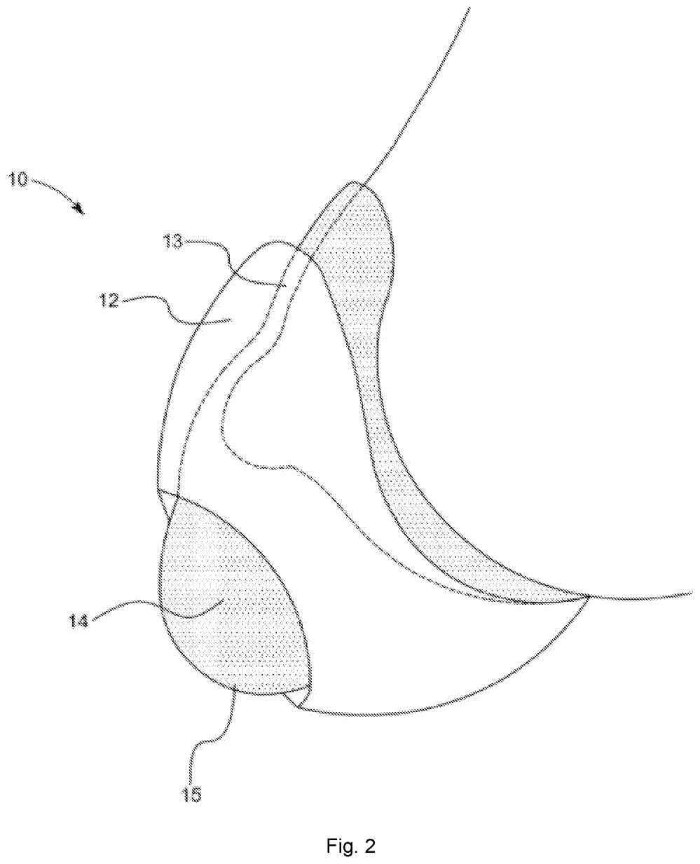

As illustrated in FIGS. 1-2, the hands-free milk extraction and collection device 10 (“the device”) includes a housing unit 12 and a milk collection container 14. The housing unit 12 has a shape contoured to a woman's breast and includes a center opening for enclosing the milk collection container 14. The housing unit is configured to latch onto the nipple region of the woman's breast to aid in stimulating milk flow from the breast by creating suction pressure at the nipple region.

The suction created by the latching of the housing unit 12 onto the nipple region of the breast is effected without the use of an electric pump and without any manual pumping. When the housing unit 12 latches onto the nipple region of the breast, a seal is created between the outer perimeter of the device 10 and the woman's breast around the nipple region. The housing unit latches onto the nipple region in a close-fitting (i.e., tight) manner which creates suction pressure by causing a squeezing and massaging effect to the nipple region. This suction pressure draws out (i.e., extracts) the milk directly from the breast and into the milk collection container 14.

The housing unit 12 may be made from any suitable material. In a preferred embodiment, the housing unit is made from plastic. The housing unit and the enclosed milk collection container are collectively configured to fit inside a woman's bra for hands free usage.

In another exemplary embodiment, the device 10 includes a plastic cap which is configured to fit inside the woman's bra to keep the pressure from the bra from releasing the suction.

The milk collection container 14 includes an internal cavity having a first section 13 for receiving the nipple region of the woman's breast and having a second section 15 for collecting the milk extracted from the woman's breast. The milk collection container may be sized to collect any suitable amount of milk. In a preferred embodiment, the milk collection container is able to store six to eight ounces of milk. The milk collection container may be made from any suitable material, such as, for example, plastic or silicone.

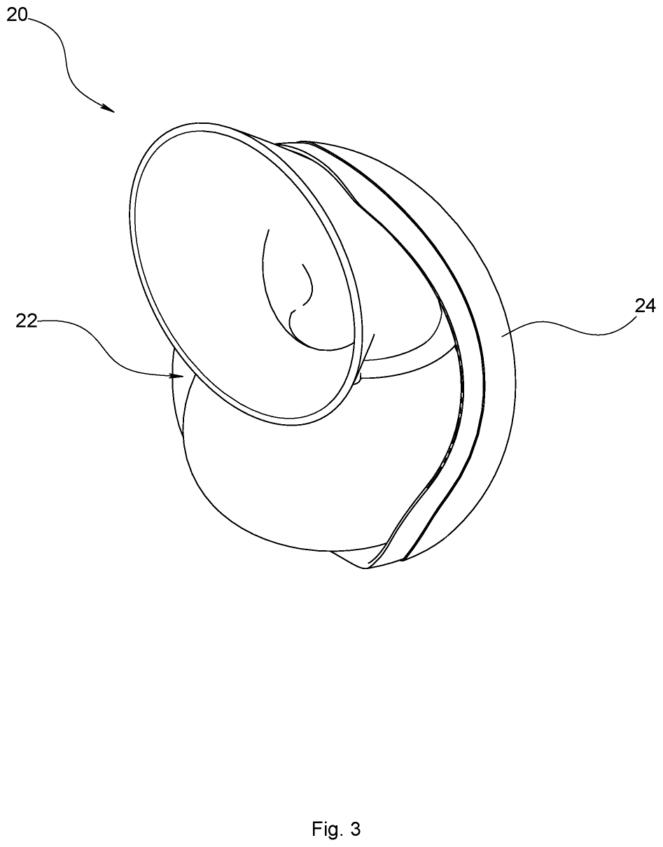

FIG. 3 shows an alternative embodiment of a hands-free milk extraction and collection device 20. The milk extraction and collection device 20 includes a combined extraction and collection container 22 and a housing unit 24.

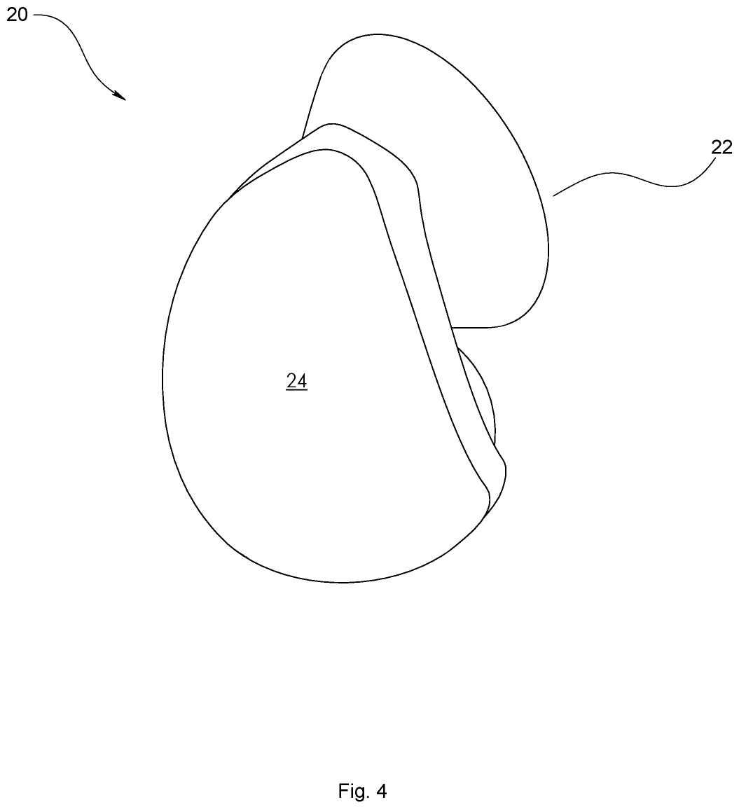

FIG. 4 shows the device from a rear perspective to give a better view of the housing unit 24. The combined extraction and collection container 22 should be made of a relatively flexible material, for example, as flexible plastic, silicone, a treated fabric, etc., that should provide a relatively comfortable close-fitting seal which would be used to create suction over the user's nipple area of the breast. The housing unit 24 can be of a relatively stiffer material, such as a hard plastic.

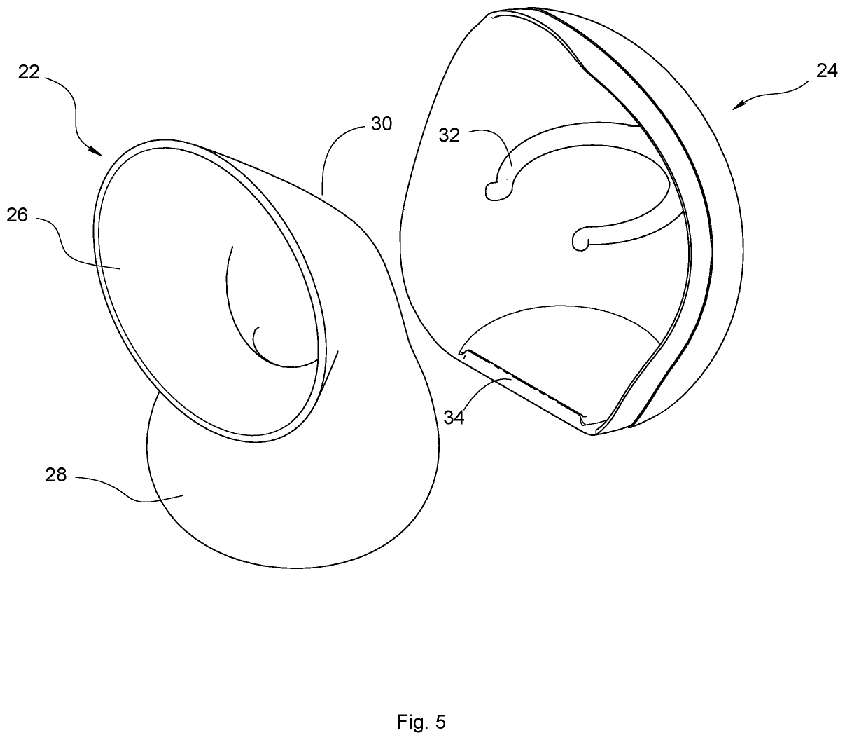

FIG. 5 shows the combined extraction and collection container 22 separated from the housing unit 24. The combined extraction and collection container may include an extraction portion 26 for engaging with the user's breast and a collection portion 28 for retained the expressed milk. The extraction portion 26 and collection portion 28 may be separated by a neck portion 30.

The interior of the housing may contain a clip portion 32 for engaging with the neck portion 30 to securely retain the combined extraction and collection container 22 in the housing unit. A ridge portion 34 at the front lip of the housing unit may also be provided to help retain the container 22 in the housing unit 24.

The clip portion 32 may be removable or fixed to the housing unit 24. The clip portion may be rigid, or have a slight bending “give” to ease insertion of the container 22 into the housing 24. In alternative embodiments, other retaining devices, such as a nylon belt, may be connected to the housing for securing the neck portion 30 or other portions of the container. 22.

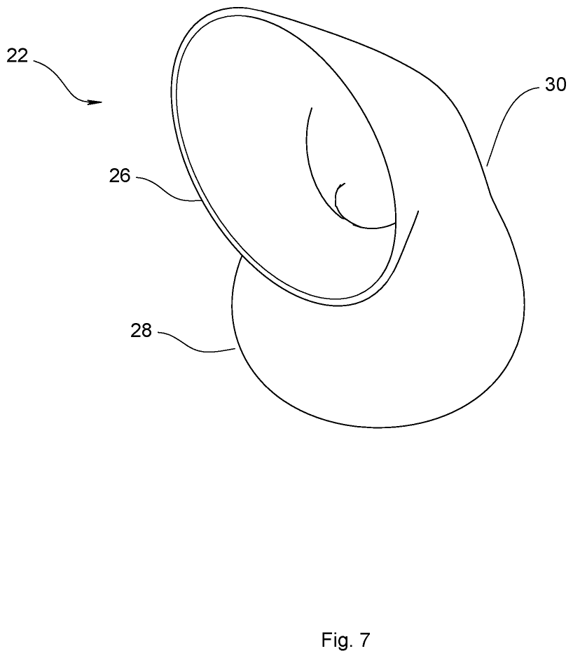

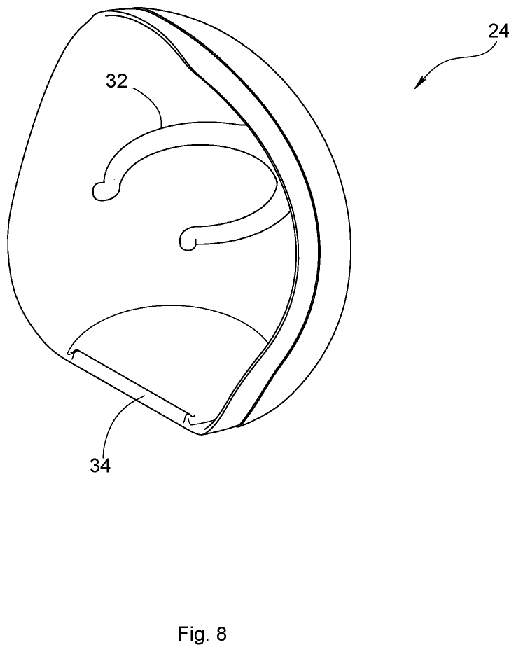

FIGS. 7 and 8 show individual views of the container 22 and housing unit 24, respectively.

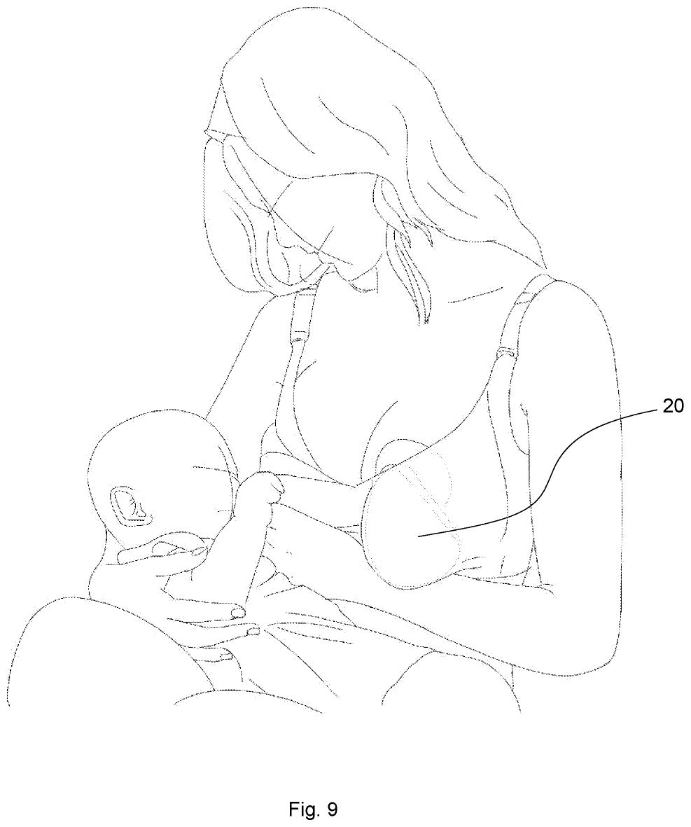

FIG. 9 shows a user using the device 20 with the device 20 in the cup of the user's bra for the non-feeding breast for hands-free use.

Accordingly, while example embodiments are capable of various modifications and alternative forms, embodiments thereof are shown by way of example in the figures and are herein described in detail. It should be understood, however, that there is no intent to limit example embodiments to the particular forms disclosed, but on the contrary, example embodiments are to cover all modifications, equivalents, and alternatives falling within the scope of the disclosure.

Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.)

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

Those skilled in the art will appreciate from the foregoing description that the broad techniques of the embodiments of the present invention may be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. A milk extraction and collection device comprising:
a housing unit, the housing unit including a retention device; and
an extraction and collection container including
an extraction portion,
a collection portion, and
a neck portion interposed between the extraction portion and the collection portion, wherein the retention device is adapted to engage at least the neck portion of the extraction and collection container.

2. The device of claim 1, wherein the extraction portion comprises a shape adapted to closely engage with a nipple region of a human breast.

3. The device of claim 2, wherein extraction and collection container comprises silicone.

4. The device of claim 1, wherein the retention device comprises a clip portion.

5. The device of claim 4, wherein the housing unit has the shape of a plastic shell including a lip, and wherein the retention device further comprises a ridge portion on the lip.

6. The device of claim 1, wherein the housing comprises plastic.
